(19) United States
(12) Patent Application Publication
(10) Pub. No.: US 2018/0193048 A1
(43) Pub. Date: Jul. 12, 2018

(54) BIFURCATED FORCEPS

(71) Applicant: HASAN ALSHEMARI, SAAD AL ABDULLA (KW)

(72) Inventor: HASAN ALSHEMARI, SAAD AL ABDULLA (KW)

(21) Appl. No.: 15/658,142

(22) Filed: Jul. 24, 2017

Related U.S. Application Data
(63) Continuation of application No. 15/404,173, filed on Jan. 11, 2017, now abandoned.

(51) Int. Cl. A61B 17/28

(52) U.S. Cl. CPC A61B 17/282

(57) ABSTRACT

The bifurcated forceps includes two pairs of forceps arms resiliently joined at a pivot end of the arms, one arm in each pair being linked to a corresponding arm in the other pair so that the first pair of corresponding arms can be rotated to a spaced radial position from the second pair of corresponding arms, enabling the corresponding forceps arms to grasp a common object at spaced apart positions. The bifurcated forceps may be used for tissue stabilization, and in non-medical applications.

### CROSS-REFERENCE TO RELATED APPLICATION

This application claims the benefit of U.S. patent application Ser. No. 15/404,173 filed on Jan. 11, 2017, presently pending.

### BACKGROUND OF THE INVENTION

### 1. Field of the Invention

A set of forceps with two sets of jaws which can be adjusted relative to each other over a range of angles The primary use for the forceps is stabilization of tissue. The forceps can be used across a wide range of medical and non-medical applications where gripping at two points is desirable.

### 2. Description of the Related Art

Common forceps are designed to have a single pair of gripping arms which extend from a rear connection point and terminate at the same relative position. This allows for an object to be gripped between the two terminal ends. Generally, the terminal points come to a fine point or pad to allow for delicate manipulation however, the terminal ends can be of any shape or size. The terminal ends may have different gripping geometry to allow for gripping difficult or slippery objects. Common applications for these types of forceps include but are not excluded to, medical gripping, sport fishing and cosmetic. If an instance arises where an object must be gripped at two locations for purposes such as but not limited to Stabilization, a second set of forceps must be used requiring a second hand which may be from the first user or from another individual.

### SUMMARY OF THE INVENTION

Forceps have been designed which bifurcate the terminal ends into two sets of gripping points which results in four terminal points extending from a common attachment point. The terminal ends are designed to contact in pairs such that upon the user pinching the device together, the two pairs of terminal ends with contact the respective mate allowing for two points of gripping. The primary downside to this design is that the sets of terminal ends are fixed in distance relative to each other. If a situation arise where a wider or shallower gripping distance is required, a different set of forceps would be required.

### BRIEF DESCRIPTION OF THE DRAWINGS

FIGS. 1-10 are various views of the bifurcated forceps.

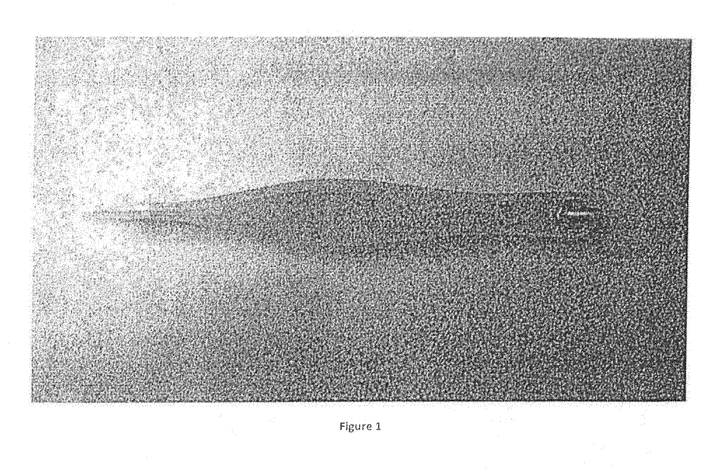

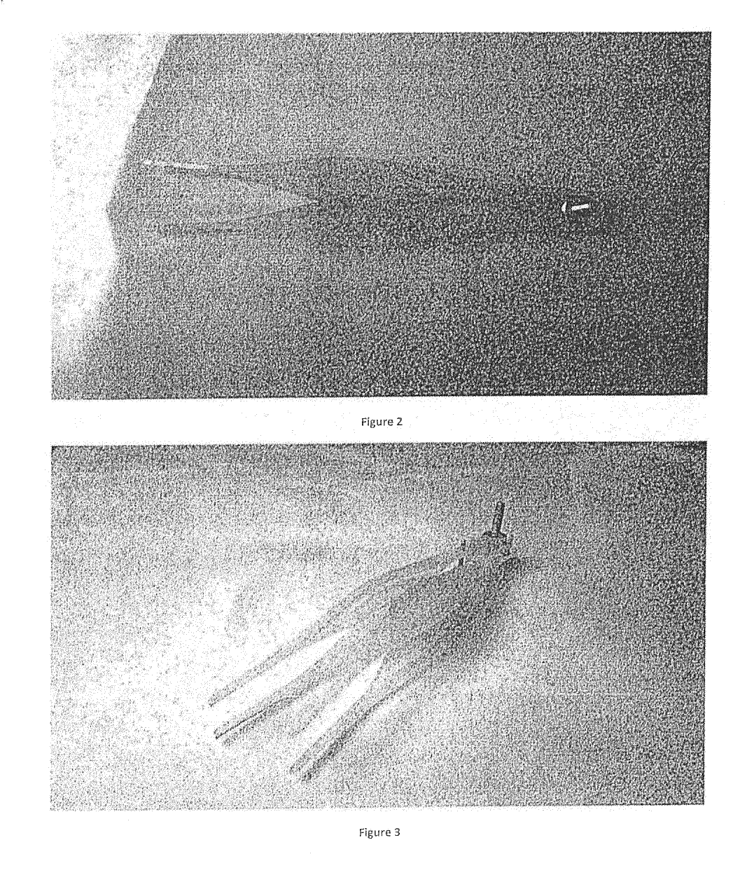

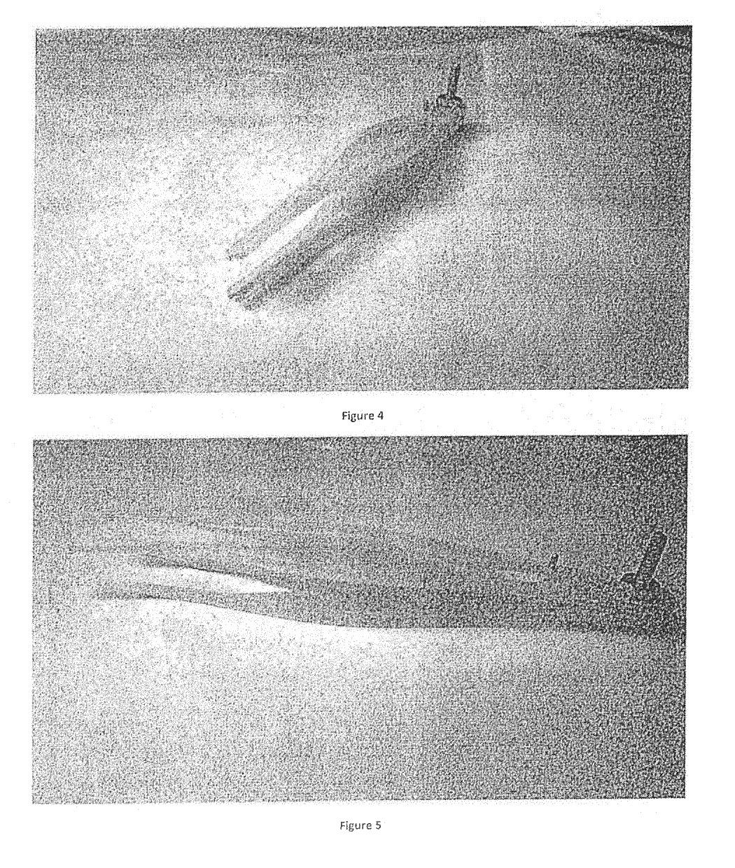

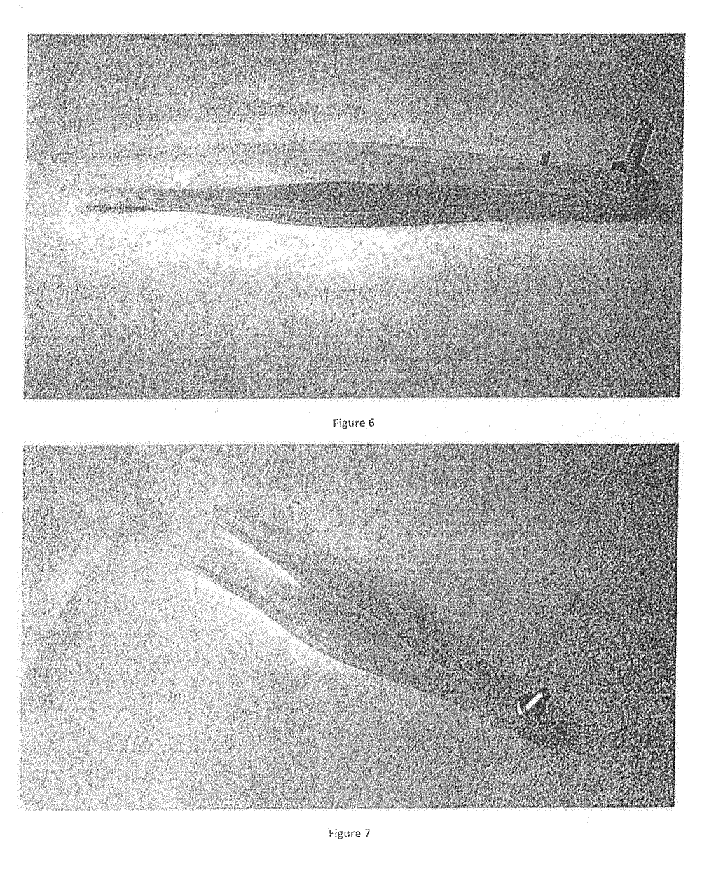

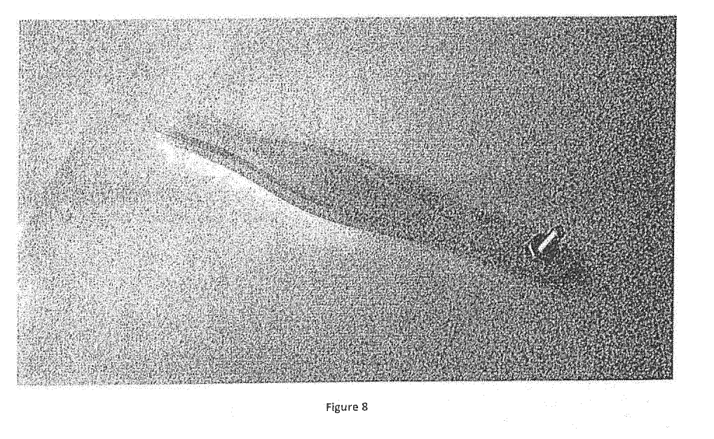

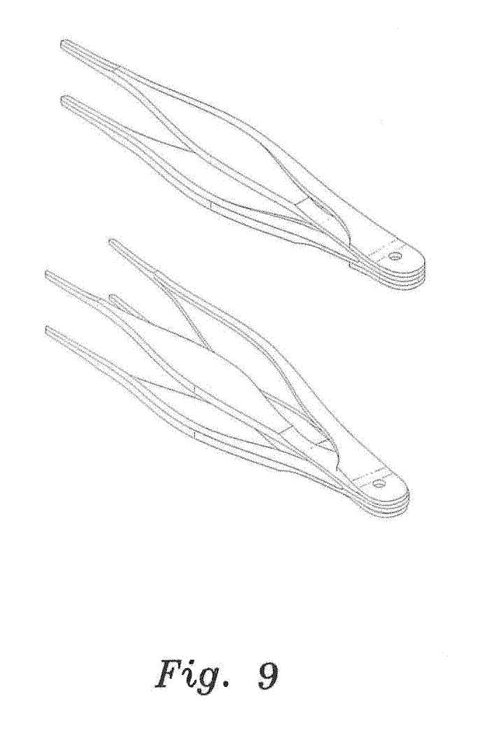

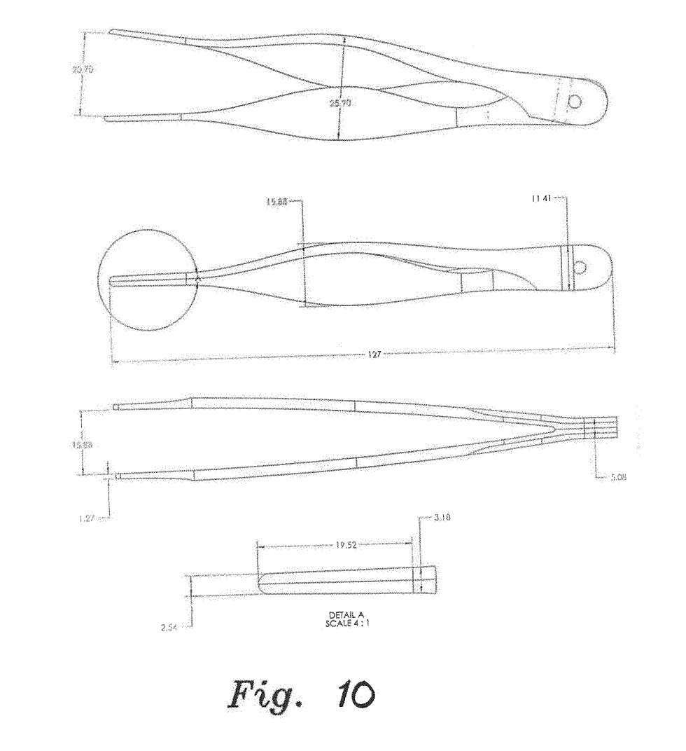

### DETAILED DESCRIPTION OF THE PREFERRED EMBODIMENTS

Forceps have been designed which bifurcate the terminal ends into two sets of gripping points which results in four terminal points extending from a common attachment point. The terminal ends are designed to contact in pairs such that upon the user pinching the device together, the two pairs of terminal ends with contact the respective mate allowing for two points of gripping. The primary downside to this design is that the sets of terminal ends are fixed in distance relative to each other. If a situation arise where a wider or shallower gripping distance is required, a different set of forceps would be required.

This embodiment of bifurcated forceps aims to address the spacing issue. The device is designed to allow a user to selectively adjust the distance between the terminal end pairs across a range of distances.

The current embodiment consists of a four relatively equal in length arms. The arms are grouped in two gripping pairs which generally mirror each other respective to the gripping plane. The mirrored sets of gripping pairs are fixed to their coordinating gripping mate on the end opposite the gripping ends. Both sets of gripping pairs are attached to one another by means of a pivot which allows one gripping pair to move relative to other while keeping the terminal end of each gripping pair aligned such that when forced together, the gripping features will contact at relatively the same point.

It is not required, but was found to be desirable to have the lengths of the gripping pairs which mirror the gripping plane possess geometry which overlap the two lengths. This allowed for both lengths on each side to be forced together in tandem when acted upon at a single point.

It is to be understood that the present invention is not limited to the embodiments described above, but encompasses any and all embodiments within the scope of the following claims.

I claim:

1. A bifurcated forceps, comprising a first pair of forceps arms and a second pair of forceps arms, each of the arms having a pivot end and a gripping end, the pivot ends of the first and second pairs of forceps arms being resiliently joined to each other at the pivot ends, the first and second pairs of forceps arms being biased to an open position in which the gripping ends are separated from each other and resiliently pivotal towards each other in a gripping plane in order to grip an object between the gripping ends, the pivot ends of the two arms in each of the pairs being pivotal around a common pivot axis in a rotational plane orthogonal to the gripping plane, each of the arms in the first pair being linked to and constrained to rotate to a common radial angle with a corresponding one of the arms in the second pair to define two corresponding pairs of forceps arms, whereby the corresponding pairs of forceps arms may be rotated to spaced radial positions in order to grip the same object at spaced apart positions.
